(19) United States
(12) Plant Patent Application Publication
(10) Pub. No.: US 2013/0104268 P1
(43) Pub. Date: Apr. 25, 2013

(54) Grape plant named 'ARRANINETEEN'

(50) ;

(71) Applicants: Sal Giumarra, Edison, CA (US); Shachar Karniel, Edison, CA (US)

(72) Inventors: Sal Giumarra, Edison, CA (US); Shachar Karniel, Edison, CA (US)

(21) Appl. No.: 13/317,677

(22) Filed: Oct. 24, 2011

(51) Int. Cl. A01H 5/00

(52) U.S. Cl. PLT/205

(57) ABSTRACT

A new distinct variety of grapevine named ‘ARRANINETEEN’ abundantly forms attractive medium-to-large seedless berries having a pink/red skin coloration in medium-to-large clusters. The fruit displays a sweet crisp flavor and is firm in texture. The fruit commonly is ready for harvesting during July in San Joaquin Valley of Central California, U.S.A, and displays good eating qualities as a table grape. The fruit firmness renders the fruit well amenable for handling, shipping, and storage.

### CLASSIFICATION

The present invention relates to a new Vitis vinifera Grapevine.

### VARIETY DENOMINATION

The new Grapevine has the varietal denomination ‘ARRANINETEEN’

### BACKGROUND OF THE INVENTION

A breeding program was initiated during the late 90′s near Bakersfield in the San Joaquin Valley of Central California. In 2001, during this breeding program, a new variety of Vitis vinifera was created by deliberate cross breeding of two parent plants by emasculation of the pollen bearing organ of the male and introducing pollen from another male origin. The female parent of the new variety was the GAR4 which is a dark red, elongated, meaty grape variety with a very small seed trace and low sugar content (non-patented in the United States). The male parent (i.e. the pollen parent) of the new variety was GZR1 which is a round, pinkish grape variety (non-patented in the United States).

Comparison between ARRANINETEEN and Red Globe

| | ARRANINETEEN | Red Globe |
|---|---|---|
| Vigor | Strong | Vigorous |
| Berry Color | Red | Grayish red |
| Berry shape | Broad Ellipsoid | Globose |
| Seed trace | Rudimentary | None |
| Flavor | None | Naturally sweet |

The parentage of the new variety can be summarized as follows:

GAR4×GZR1

An artificial pollination was created, and the result was an embryo which possessed unique genetic qualities. The rudiments resulting from the above pollination were embryo rescued.

In 2002 the plant was transplanted to Bakersfield in San Joaquin Valley of Central California.

It was found that the new grapevine of the present invention possesses the following combination of the characteristics:

(a) Forms attractive medium-to-large seedless berries with a pink/red skin coloration which display a natural flavor;

(b) Commonly bears fruit during the month of July in the San Joaquin Valley of Central California, U.S.A; and

(c) Bears fruit that is firm and well amenable for storage, handling and shipping.

The new variety during observation to date has displayed no visible disease, and has displayed an ability to well resist cold, drought, heat; but sensitive to direct exposure to sun and wind. The fruit of the new variety has been found to display excellent handling and shipping qualities combined with desirable dessert eating qualities.

The new variety of the present invention has been found to undergo asexual propagation beginning in 2005 near Bakersfield in the San Joaquin Valley of Central California, U.S.A. by grafting on mature ‘Thompson Seedless’ rootstock (non-patented in the United States). Such asexual propagation has been conducted thereafter in successive years through 2008, and has shown that the characteristics of the new variety are strictly transmissible from one generation to another. Accordingly, the new variety undergoes asexual propagation in a true to type manner.

### SUMMARY OF THE INVENTION

The new variety ‘ARRANINETEEN’ is a round, full, meaty and sweet grape variety which bears a slight seed rudiment with medium to large production, e.g., about 40 to 50 bunches per vine, and an average of about one to two bunches per shoot.

Asexual reproduction by micro propagation of the new variety as performed near Bakersfield, Calif., U.S.A., at Arvin, Calif. Ranch 33 which shows that the forgoing and other distinguishing characteristics come true to form and are established and transmitted through succeeding propagations.

### BRIEF DESCRIPTION OF THE DRAWING

The accompanying photographic illustration shows typical specimens of vegetative growth of five year old specimens of the new variety, in color as nearly true as it is reasonably possible to make in a color illustration of this character. Colors in the photograph may differ from the color values cited in the detailed botanical description below, which accurately describes the colors of the new Grapevine.

FIG. 1 shows leaves, stems and grapes of ‘ARRANINETEEN’

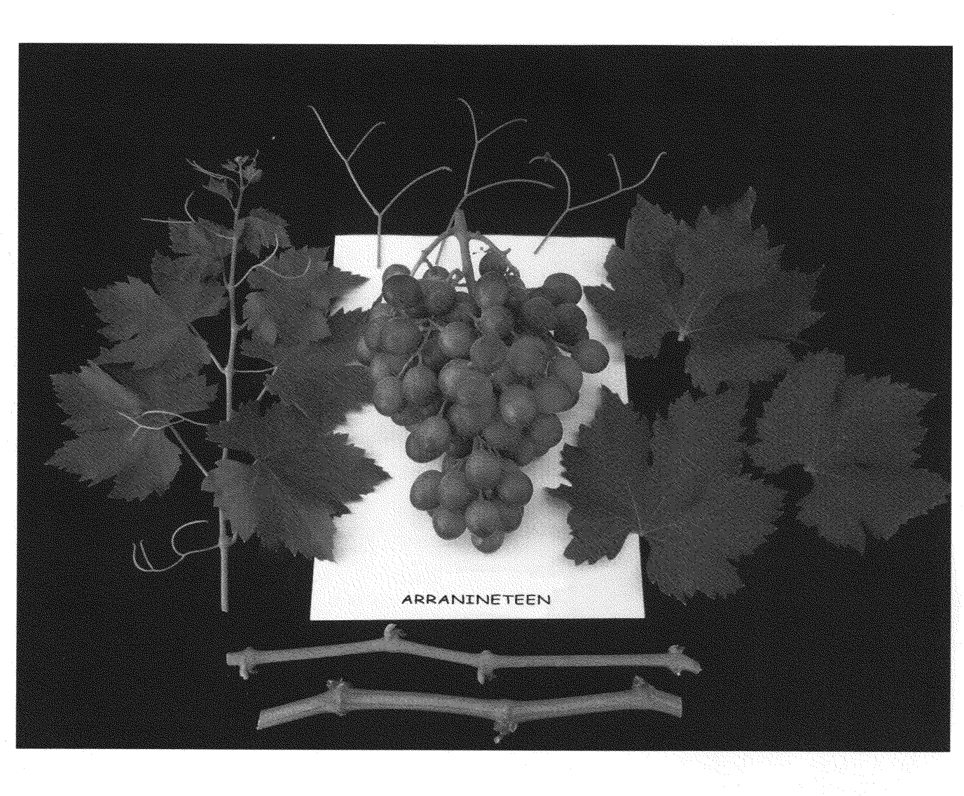

### DETAILED BOTANICAL DESCRIPTION

The chart used in the identification of colors is the R.H.S. Colour Chart of THE ROYAL HORTICULTURAL SOCIETY. The description is based on the observation of plants growing on ‘Thompson Seedless’ rootstock outdoors near San Joaquin Valley of Central California, U.S.A.
Vine:
   Vigor.—Very strong vine, large crop, good foliage coverage.
   Productive capacity.—23-35 ton per hectare.
   Trunk.—Strong and developed. Ranges from 2″- 2″ ⅜″. Color of the trunk is greyed-orange (166-B).
Time of bud burst:
   Early.—Date of bud burst in specified location of culture is Mar. 23, 2011 in Arvin, Calif.
Young shoot:
   Openness of tip.—Slightly open.
   Prostrate hairs on tip.—sparse.
   Anthocyanin coloration of prostate hairs on tip.—weak.
   Erect hairs on tip.—Sparse.
Young leaf:
   Color of upper side of blade.—yellow-green (152-A).
   Prostrate hairs between main veins on lower side of blade.—medium.
   Erect hairs on main veins on lower side of blade.—medium.
Shoot:
   Attitude (before tying).—Semi-erect.
   Color of dorsal side of internodes.—yellow green (144-B).
   Color of ventral side of internodes.—Yellow green (144-C).
   Color of dorsal side of nodes.—Yellow green (144-B).
   Color of ventral side of nodes.—Yellow Green (144-C).
   Erect hairs on internode.—absent or very sparse.
   Length of tendrils.—8″.
   Color of tendrils.—Yellow Green (145-A).
Flower:
   Sexual organs.—fully developed stamens and fully developed gynoecium.
Mature leaf:
   Size of blade.—6×7.5″.
   Shape of blade.—circular.
   Blistering upper side of blade.—absent or very weak.
   Number of lobes.—five.
   Depth of upper lateral sinuses.—absent or very shallow.
   Only varieties with lobed leaves.—mixture of both sides straight and both sides convex.
   Arrangement of lobes of upper lateral sinuses.—closed.
   Arrangement of lobes petiole sinus.—slightly open.
   Length of teeth.—Medium; length 0.4″, width 0.35″.
   Ratio length/width of teeth.—medium.
   Shape of teeth.—mixture of both sides straight and both sides convex.
   Proportion of main veins on upper side of blade with anthocyanin coloration.—medium.
   Prostrate hairs between main veins on lower side of blade.—absent or very sparse.
   Erect hairs on main veins on lower side of blade.—absent or very sparse.
   Length of petiole equal compared to length of middle vein.—moderately shorter.
   Top side color.—Green (137-A).
   Bottom side color.—Yellow green (146-B).
   Texture.—Smooth.
   Vein color.—Yellow green (145-C).
   Petiole length.—4.7″.
   Petiole color.—Green (144-C).
   Petiole strips color.—Grey red (182-C).
Reproductive organs:
   Color.—Green (143-A).
   Size.—0.07″.
Time of beginning of ripening: Aug. 15th Arvin, Calif. season
Bunch:
   Size (penduncle excluded).—Large 9×8″.
   Density.—Medium.
   Length of penduncle primary bunch.—medium 7.5″.
   Average bunch weight.—700 grams.
Berry:
   Size.—Large 14/16.
   Shape.—broad ellipsoid.
   Weight.—8 grams.
   Color of skin (without bloom).—greyed purple (183-C).
   Ease of detachment from pedicle.—Difficult.
   Thickness of skin.—Thin.
   Anthocyanin coloration of flesh.—White (155-D).
   Firmness of flesh.—very firm.
   Particular flavor.—None.
   Formation of seeds.—Rudimentary.
   Market use of the observed plant.—Fresh market.
   Berries per bunch.—100 berries.
Woody shoot:
   Main color.—Greyed orange 164-B.
DNA Profile: To further the characterize the new Arra variety DNA was extracted from dried leaf samples and DNA profiles were obtained in California, using base pairs for 8 standard microsatellite DNA markers. The data is presented hereafter.

| Microsatellite | Allele Sizes in | |
|---|---|---|
| DNA Marker | Base Pairs | |
| MSV01 | 132 | 152 |
| MSV02 | 234 | 236 |
| MSV04 | 179 | 193 |
| MSV06 | 254 | 258 |
| MSV07 | 318 | 318 |
| MSV08 | 245 | 251 |
| MSV09 | 241 | 251 |
| MSV10 | 211 | 211 |
| MSV12 | 246 | 259 |
| MSV13 | 168 | 168 |
| MSV14 | 162 | 180 |
| MSV15 | 291 | 295 |
| MSV16 | 188 | 188 |
| MSV17 | 159 | 159 |

What is claimed is:

1. A new and distinct variety of grapevine, botanically known as Vitis vinifera, identified as ‘ARRANINETEEN’, substantially as shown and described herein.
